(19) United States
(12) Patent Application Publication
(10) Pub. No.: US 2020/0129418 A1
(43) Pub. Date: Apr. 30, 2020

(54) Chinese Medical Cosmetic Composition

(71) Applicant: Shanghai Pinmai Inter Tech Co., Ltd, Shanghai (CN)

(72) Inventor: Wen Liu, Shanghai (CN)

(21) Appl. No.: 16/175,702

(22) Filed: Oct. 30, 2018

(51) Int. Cl. A61K 8/9789; A61K 36/258; A61K 36/886; A61K 36/355; A61K 36/63; A61K 36/288; A61K 8/06; A61K 8/92; A61Q 19/02; A61Q 17/04; A61Q 19/00

(52) U.S. Cl. CPC A61K 8/9789; A61K 36/258; A61K 36/886; A61K 36/355; A61K 36/63; A61K 36/288; A61K 2236/51; A61K 8/922; A61Q 19/02; A61Q 17/04; A61Q 19/007; A61K 2236/31; A61K 2236/333; A61K 8/062

(57) ABSTRACT

A Chinese medical cosmetic composition comprising Radix Notoginseng extract, Aloe extract and Flos Lonicerae extract. The Chinese medical cosmetic composition has a scientific and reasonable formula composition, and has the effects of tonifying Qi and nourishing Yin, promoting blood circulation and removing blood stasis, moisturizing and whitening, and strengthening body and sun screening.

### TECHNICAL FIELD

The present invention relates to the field of cosmetic and medicine, in particular to a Chinese medical cosmetic composition.

### BACKGROUND

All the people want to possess a healthy and fair skin.

In order to prevent pigmentation, the basic principle of whitening cosmetics is mainly embodied in the following aspects: 1. Inhibition of melanin production, wherein the melanin production is prevented by inhibiting tyrosinase activity or interfering with melanin-producing intermediates. 2. Reduction of melanin and prevention of photooxidation, wherein the melanin reduction is stimulated by keratinocytes, so as to remove the produced melanin. 3. Reduction of pigmentation during skin metabolism, wherein, the melanin is quickly discharged from the skin by improving the metabolism of the skin. 4. Prevention of the entry of UV light, wherein, the excessive melanin caused by UV rays is reduced by physically blocking UV rays with a sun-screening preparation.

The current whitening products are mainly used to inhibit the activity of tyrosinase. Although there are many health-care cosmetics, for various reasons, their utilization is not satisfactory and cannot satisfy the practical application. Therefore, the improvement and innovation for the cosmetics are imperative.

The skin is a large-area human-body organ which is exposed to the outside, and the water in the cells and the matrix of the skin is easily to be lost. There is no blood vessel in the epidermis of the skin, and the water may only be supplied by the penetration through the dermis, thus the epidermis is the part of the human body which is most easily subjected to dehydration. Loss of water will affect the skin cell division and secretion ability, reduce pigment metabolism and reduce moisturizing effect on itself. Effectively replenishing and retaining the water of the skin is an important nonnegligible aspect of reducing pigmentation in the skin. Therefore, keeping the skin moisturized is the basis of removing freckle and whitening.

### SUMMARY OF THE INVENTION

The present invention relates to a Chinese medical cosmetic composition, which can effectively solve the problems of cosmetics used for freckle removing, moisturizing and whitening.

The technical solution of the present invention is as follows:

The Chinese medical cosmetic composition is prepared by mixing the following raw materials, by weight percentage, Radix Notoginseng extract 5-10%, Aloe extract 0.1-1%, Flos Lonicerae extract 5-10%, dandelion extract 5-20%, ascorbic acid (vitamin C) 2-3%, olive oil 12-17%, isopropyl palmitate 4-6%, polyoxyethylene nonylphenol ether 0.4-0.6%, glycerol 4-6%, methyl p-hydroxybenzoate 0.08-0.12%, ethanol 6-8%, and deionized water in balance (i.e. the deionized water is added until the total percentage is 100%),

wherein,

the Radix Notoginseng extract is prepared by the following steps: heat refluxing Radix Notoginseng with 50 mass % ethanol as a solvent twice, each time with an ethanol/Radix Notoginseng weight ratio of (6-8):1 for 1 h, to obtain two ethanol extraction solutions; combining the two ethanol extraction solutions, and recovering the ethanol from the resultant for concentration to obtain an extractum with a relative density of 1.2-1.4; and freezing the extractum at −80° C. for 24 h, and vacuum drying to obtain freeze-dried powder as the Radix Notoginseng extract;

the Aloe extract is prepared by the following steps: heat refluxing Aloe with 70 mass % ethanol as a solvent twice, each time with an ethanol/Aloe weight ratio of (6-8):1 for 1 h, to obtain two ethanol extraction solutions; combining the two ethanol extraction solutions, and recovering the ethanol from the resultant for concentration to obtain an extractum with a relative density of 1.2-1.4; and freezing the extractum at −80° C. for 24 h, and vacuum drying to obtain freeze-dried powder as the Aloe extract;

the Flos Lonicerae extract is prepared by the following steps: heat refluxing Flos Lonicerae with 50 mass % ethanol as a solvent twice, each time with an ethanol/Flos Lonicerae weight ratio of (6-8):1 for 1 h, to obtain two ethanol extraction solutions; combining the two ethanol extraction solutions, and recovering the ethanol from the resultant for concentration to obtain an extractum with a relative density of 1.2-1.4; and freezing the extractum at −80° C. for 24 h, and vacuum drying to obtain freeze-dried powder as the Flos Lonicerae extract;

and the dandelion extract is prepared by the following steps: heat refluxing dandelion with 90 mass % ethanol as a solvent twice, each time with an ethanol/dandelion weight ratio of (6-8):1 for 1 h, to obtain two ethanol extraction solutions; combining the two ethanol extraction solutions, and recovering the ethanol from the resultant for concentration to obtain an extractum with a relative density of 1.2-1.4; and freezing the extractum at −80° C. for 24 h, and vacuum drying to obtain freeze-dried powder as the dandelion extract.

The inventive Chinese medical cosmetic composition has a scientific and reasonable formula composition, has the effects of tonifying Qi and nourishing Yin, promoting blood circulation and removing blood stasis, moisturizing and whitening, and strengthening body and sun screening, can effectively remove free radicals, inhibit the activity of tyrosinase, improve skin metabolism, and reduce the damage of the skin due to UV rays, and is natural and non-irritating, so as to achieve the effect of fully whitening skin.

### DETAILED DESCRIPTION OF THE EMBODIMENTS

Hereafter, the specific embodiments of the present invention will be explained in combination with the examples.

The present invention can be implemented by the following examples:

### EXAMPLE 1

In an embodiment of the present invention, the Chinese medical cosmetic composition is prepared by uniformly mixing the following raw materials, by weight percentage, Radix Notoginseng extract 7-8%, Aloe extract 0.4-0.6%, Flos Lonicerae extract 7-8%, dandelion extract 10-15%, ascorbic acid 2.3-2.6%, olive oil 13-16%, isopropyl palmitate 4.5-5.5%, polyoxyethylene nonylphenol ether 0.45-0.55%, glycerol 4.5-5.5%, methyl p-hydroxybenzoate 0.09-0.11%, ethanol 6.5-7.5%, and deionized water in balance.

### EXAMPLE 2

In an embodiment of the present invention, the Chinese medical cosmetic composition is prepared by uniformly mixing the following raw materials, by weight percentage, Radix Notoginseng extract 5%, Aloe extract 0.5%, Flos Lonicerae extract 5%, dandelion extract 8%, ascorbic acid 2%, olive oil 15%, isopropyl palmitate 5%, polyoxyethylene nonylphenol ether 0.5%, glycerol 5%, methyl p-hydroxybenzoate 0.1%, ethanol 7%, and deionized water in balance.

### EXAMPLE 3

In an embodiment of the present invention, the Chinese medical cosmetic composition is prepared by uniformly mixing the following raw materials, by weight percentage, Radix Notoginseng extract 9%, Aloe extract 0.7%, Flos Lonicerae extract 7%, dandelion extract 15%, ascorbic acid 2%, olive oil 15%, isopropyl palmitate 5%, polyoxyethylene nonylphenol ether 0.5%, glycerol 4%, methyl p-hydroxybenzoate 0.1%, ethanol 8%, and deionized water in balance.

### EXAMPLE 4

In an embodiment of the present invention, the Chinese medical cosmetic composition is prepared by uniformly mixing the following raw materials, by weight percentage, Radix Notoginseng extract 10%, Aloe extract 1%, Flos Lonicerae extract 10%, dandelion extract 18%, ascorbic acid (vitamin C) 2.5%, olive oil 16%, isopropyl palmitate 6%, polyoxyethylene nonylphenol ether 0.4%, glycerol 6%, methyl p-hydroxybenzoate 0.11%, ethanol 8%, and deionized water in balance.

Above Chinese medical ingredients can be cooperated with other components to achieve the effect of synergistic interaction, has the effects of tonifying Qi and nourishing Yin, promoting blood circulation and removing blood stasis, moisturizing and whitening, and strengthening body and sun screening, can effectively remove free radicals, inhibit the activity of tyrosinase, improve skin metabolism, and reduce the damage of the skin due to ultraviolet rays, and is natural and non-irritating, so as to achieve the effect of fully whitening skin. The Chinese medical cosmetic composition of the present invention was tested to achieve a satisfactory advantageous technical effect, and the test data was as follows:

### 1. Inhibition of Melanophore Division

The melanin synthesis function of mouse B16 melanocytes is substantially the same as that of normal human skin melanocytes. The melanocytes were inoculated in a 96-well plate at 1×104 cells/well (100 μL per well), and cultured in a 5% CO2 incubator at 37° C. for 2 days; and the original culture solution was drawn away, and a complete culture solution was added. Above four Chinese medical extracts to be tested were mixed in proportion and diluted to obtain 5 solutions with different final medicine concentrations of 500 mg/L, 250 mg/L, 125 mg/L, 62.5 mg/L and 31.25 mg/L. For each final concentration of the solution, five parallel wells were filled; and for a control group, no medicine was added but an equal amount of complete medium was added, all of above resulted test samples were further cultured in the incubator for 3 days. The culture supernatant was removed, each well was added with 100 μL of 0.5 mg.L-1 MTT, and was further cultured for 4 h; and 150 μL of DMSO was then added to dissolve the resulted purple precipitate, and the absorbance (A) was measured by a microplate reader at a wavelength of 570 nm (/630 nm). The absorbance value of each well is divided by the average absorbance value of the normal wells, and then is multiplied by 100%, to obtain a survival rate of each well, and an inhibition rate=1−survival rate; and according to the resulted inhibition rate curve, a lethal concentration range and an IC50 medium lethal concentration of the test samples were calculated, which indicated the toxicity index of the Chinese medical whitening ingredients (see Table 1);

TABLE 1
Inhibition rate of melanocytes

| | | | | | | |
|---|---|---|---|---|---|---|
| Medicine | 500 | 250 | 125 | 62.5 | 31.25 | Control |
| concentration | | | | | | |
| (mg/L) | | | | | | |
| Inhibition rate % | 64.10 ± 8.32^{▴} | 49.66 ± 7.02^{▴} | 31.16 ± 4.82^{▴} | 17.54 ± 3.45^{▴} | 11.91 ± 2.05^{▴} | 4.13 ± 0.84^{▴} |

Note:
compared with the control group: ^{▴}P < 0.05

With the concentration increase of the Chinese medical whitening ingredients, the inhibition rate of melanocytes was correspondingly increased, and the toxicity index is 250 mg/ml, that is, the concentration of the Chinese medical whitening ingredients in a whitening skin cream cannot exceed this concentration of 250 mg/ml.

### 2. Inhibition of Tyrosinase Activity

After the Chinese medical whitening ingredients (at above concentrations) have functioned for 3 days, the supernatant was discarded, and each well was washed twice with pH 6.8 PBS and added with 90 μL of PBS containing 1% (by volume percentage) of Triton X-100. Each well was subjected to ultrasonic crushing in an ice bath, and added with 10 uL of 1×10-2 mol/L L-dopa, and then incubated at 37° C. for 60 min; and a UV spectrophotometer was used for colorimetry at 490 nm while zeroing with a blank well, to measure the absorbance value of each well (See Table 2). Tyrosinase activity inhibition rate=(1−average absorbance value of each concentration/average absorbance value of the control group)*100%

TABLE 2
Tyrosinase activity inhibition rate

| | Medicine | | | | |
|---|---|---|---|---|---|
| | concentration (mg/L) | | | | |
| | 500 | 250 | 125 | 62.5 | 31.25 |
| Inhibition rate % | 37.98 | 32.14 | 26.34 | 19.26 | 5.94 |

It can be seen from Table 2 that, with the concentration decrease of Chinese medical whitening ingredients, the inhibitory effect on tyrosinase activity was also reduced, that is, with the concentration increase of the medicines, the inhibitory effect thereof was also improved, thus the inhibition effect on the tyrosinase activity was confirmed.

### 3. Sun Screening Effect of Whitening Skin Cream

Five rats (with a body weight of 400 g-450 g) were taken for testing. After each rat was anesthetized with diethyl ether, 3 cm×3 cm of hair at the symmetric parts on two sides of the rat back was carefully cut, and then the rat was placed in a rat cage and was allowed to move freely under UV sterilamps (25 W per sterilamp, at a distance of 100 cm), an appropriate amount of whitening skin cream was uniformly applied on the right-side skin of the rat back, and the left-side skin of the rat back was only applied thereon with a cream containing no Chinese medical ingredients; each rat was irradiated under the UV sterilamp for 8 hours every day (at the time of 12 h, each rat was anesthetized again and applied with the whitening skin cream once); and the rats were taken out, further feed for 7 days and then sacrificed. The surface of the rat skin was gently wiped with a cotton ball alternately impregnated with water and ethanol to remove the residual cream, and the thickness of the rat skin was measured with a vernier scale.

TABLE 3
Measurement of skin thickness on left and right sides of the rat (unit: um)

| | Number | | | | |
|---|---|---|---|---|---|
| | 1 | 2 | 3 | 4 | 5 |
| Left side | 48.6 | 42.9 | 45.6 | 50.1 | 47.8 |
| Right side | 41.9 | 38.2 | 44.0 | 40.1 | 39.7 |

UV damage may increase the thickness of the epidermal layer cells, UV rays will not only damage the outermost skin cells, but also reach the dermal cells through the epidermis, resulting in that necrosis and metabolic disorders of a large number of dermal cells occur, and the impurities in the cells cannot be metabolized, such that the cells lose elasticity, prematurely age, and become sensitive, and then wrinkles appear and the thickness increases. As can be seen from the data in Table 3, the right-side skin is thinner than the left-side skin, indicating that, the skin on the right side of the rat back which was applied with the whitening skin cream was protected to some extent.

### 4. Moisturizing Test of the Whitening Skin Cream

An in vitro weighing method (with reference to the weighing method used in the “Skin science and cosmetic efficacy evaluation” compiled by Liu Wei and Zhang Huailiang) is used. 5 g of whitening skin cream and 5 g of matrix were uniformly applied on two culture dish bottle caps respectively, then the culture dish bottle caps were placed for 8 h, wherein, the water reduction amount thereof was obtained by weighing every 1 h, and the water retention capacity was calculated by: residual water mass/sample mass.

TABLE 4
Water retention capacity test of the whitening skin cream within 8 h

| | | | | | | | | |
|---|---|---|---|---|---|---|---|---|
| Time interval | 1 | 2 | 3 | 4 | 5 | 6 | 7 | 8 |
| (h) | | | | | | | | |
| Water | 98.24 | 90.84 | 89.34 | 87.12 | 86.51 | 79.24 | 60.46 | 57.12 |
| retention | | | | | | | | |
| capacity | | | | | | | | |

As can be seen from Table 4, as time went by, the amount of residual water was reduced gradually. After 8 hours, the water retention capacity can be maintained at more than half, indicating that, a good moisturizing effect was achieved.

In summary, it can be clearly confirmed from the above tests that the Chinese medical cosmetic composition of the present invention has the effects of removing freckle, moisturizing and sun screening, and can be effectively used for protection and health care of human skin.

1. A cosmetic composition, prepared by mixing the following raw materials, by weight percentage, Radix Notoginseng extract 5-10%, Aloe extract 0.1-1%, Flos Lonicerae extract 5-10%, dandelion extract 5-20%, ascorbic acid 2-3%, olive oil 12-17%, isopropyl palmitate 4-6%, polyoxyethylene nonylphenol ether 0.4-0.6%, glycerol 4-6%, methyl p-hydroxybenzoate 0.08-0.12%, ethanol 6-8%, and deionized water in balance,
wherein,
the Radix Notoginseng extract is prepared by the following steps: heat refluxing Radix Notoginseng with 50 mass % ethanol as a solvent twice, each time with an ethanol/Radix Notoginseng weight ratio of (6-8):1 for 1 h, to obtain two ethanol extraction solutions; combining the two ethanol extraction solutions, and recovering the ethanol from the resultant for concentration to obtain an extractum with a relative density of 1.2-1.4; and freezing the extractum at −80° C. for 24 h, and vacuum drying to obtain freeze-dried powder as the Radix Notoginseng extract;
the Aloe extract is prepared by the following steps: heat refluxing Aloe with 70 mass % ethanol as a solvent twice, each time with an ethanol/Aloe weight ratio of (6-8):1 for 1 h, to obtain two ethanol extraction solutions; combining the two ethanol extraction solutions, and recovering the ethanol from the resultant for concentration to obtain an extractum with a relative density of 1.2-1.4; and freezing the extractum at −80° C. for 24 h, and vacuum drying to obtain freeze-dried powder as the Aloe extract;
the Flos Lonicerae extract is prepared by the following steps: heat refluxing Flos Lonicerae with 50 mass % ethanol as a solvent twice, each time with an ethanol/Flos Lonicerae weight ratio of (6-8):1 for 1 h, to obtain two ethanol extraction solutions; combining the two ethanol extraction solutions, and recovering the ethanol from the resultant for concentration to obtain an extractum with a relative density of 1.2-1.4; and freezing the extractum at −80° C. for 24 h, and vacuum drying to obtain freeze-dried powder as the Flos Lonicerae extract;
and the dandelion extract is prepared by the following steps: heat refluxing dandelion with 90 mass % ethanol as a solvent twice, each time with an ethanol/dandelion weight ratio of (6-8):1 for 1 h, to obtain two ethanol extraction solutions; combining the two ethanol extraction solutions, and recovering the ethanol from the resultant for concentration to obtain an extractum with a relative density of 1.2-1.4; and freezing the extractum at −80° C. for 24 h, and vacuum drying to obtain freeze-dried powder as the dandelion extract.

2. The cosmetic composition according to claim 1, wherein the cosmetic composition is prepared by uniformly mixing the following raw materials, by weight percentage, Radix Notoginseng extract 7-8%, Aloe extract 0.4-0.6%, Flos Lonicerae extract 7-8%, dandelion extract 10-15%, ascorbic acid 2.3-2.6%, olive oil 13-16%, isopropyl palmitate 4.5-5.5%, polyoxyethylene nonylphenol ether 0.45-0.55%, glycerol 4.5-5.5%, methyl p-hydroxybenzoate 0.09-0.11%, ethanol 6.5-7.5%, and deionized water in balance.

3. The cosmetic composition according to claim 1, wherein the cosmetic composition is prepared by uniformly mixing the following raw materials, by weight percentage, Radix Notoginseng extract 5%, Aloe extract 0.5%, Flos Lonicerae extract 5%, dandelion extract 8%, ascorbic acid 2%, olive oil 15%, isopropyl palmitate 5%, polyoxyethylene nonylphenol ether 0.5%, glycerol 5%, methyl p-hydroxybenzoate 0.1%, ethanol 7%, and deionized water in balance.

4. The cosmetic composition according to claim 1, wherein the cosmetic composition is prepared by uniformly mixing the following raw materials, by weight percentage, Radix Notoginseng extract 9%, Aloe extract 0.7%, Flos Lonicerae extract 7%, dandelion extract 15%, ascorbic acid 2%, olive oil 15%, isopropyl palmitate 5%, polyoxyethylene nonylphenol ether 0.5%, glycerol 4%, methyl p-hydroxybenzoate 0.1%, ethanol 8%, and deionized water in balance.

5. The cosmetic composition according to claim 1, wherein the cosmetic composition is prepared by uniformly mixing the following raw materials, by weight percentage, Radix Notoginseng extract 10%, Aloe extract 1%, Flos Lonicerae extract 10%, dandelion extract 18%, ascorbic acid 2.5%, olive oil 16%, isopropyl palmitate 6%, polyoxyethylene nonylphenol ether 0.4%, glycerol 6%, methyl p-hydroxybenzoate 0.11%, ethanol 8%, and deionized water in balance.

6. A cosmetic composition, prepared by mixing the following raw materials, by weight percentage, Radix Notoginseng extract 5-10%, Aloe extract 0.1-1%, Flos Lonicerae extract 5-10%, dandelion extract 5-20%, ascorbic acid 2-3%, olive oil 12-17%, isopropyl palmitate 4-6%, polyoxyethylene nonylphenol ether 0.4-0.6%, glycerol 4-6%, methyl p-hydroxybenzoate 0.08-0.12%, ethanol 6-8%, and deionized water in balance.

7. The cosmetic composition according to claim 6, wherein the Radix Notoginseng extract is prepared by the following steps: heat refluxing Radix Notoginseng with 50 mass % ethanol as a solvent twice, each time with an ethanol/Radix Notoginseng weight ratio of (6-8):1 for 1 h, to obtain two ethanol extraction solutions; combining the two ethanol extraction solutions, and recovering the ethanol from the resultant for concentration to obtain an extractum with a relative density of 1.2-1.4; and freezing the extractum at −80° C. for 24 h, and vacuum drying to obtain freeze-dried powder as the Radix Notoginseng extract.

8. The cosmetic composition according to claim 6, wherein the Aloe extract is prepared by the following steps: heat refluxing Aloe with 70 mass % ethanol as a solvent twice, each time with an ethanol/Aloe weight ratio of (6-8):1 for 1 h, to obtain two ethanol extraction solutions; combining the two ethanol extraction solutions, and recovering the ethanol from the resultant for concentration to obtain an extractum with a relative density of 1.2-1.4; and freezing the extractum at −80° C. for 24 h, and vacuum drying to obtain freeze-dried powder as the Aloe extract.

9. The cosmetic composition according to claim 6, wherein the Flos Lonicerae extract is prepared by the following steps: heat refluxing Flos Lonicerae with 50 mass % ethanol as a solvent twice, each time with an ethanol/Flos Lonicerae weight ratio of (6-8):1 for 1 h, to obtain two ethanol extraction solutions; combining the two ethanol extraction solutions, and recovering the ethanol from the resultant for concentration to obtain an extractum with a relative density of 1.2-1.4; and freezing the extractum at −80° C. for 24 h, and vacuum drying to obtain freeze-dried powder as the Flos Lonicerae extract.

10. The cosmetic composition according to claim 6, wherein the dandelion extract is prepared by the following steps: heat refluxing dandelion with 90 mass % ethanol as a solvent twice, each time with an ethanol/dandelion weight ratio of (6-8):1 for 1 h, to obtain two ethanol extraction solutions; combining the two ethanol extraction solutions, and recovering the ethanol from the resultant for concentration to obtain an extractum with a relative density of 1.2-1.4; and freezing the extractum at −80° C. for 24 h, and vacuum drying to obtain freeze-dried powder as the dandelion extract.
